(19) United States
(12) Patent Application Publication
(10) Pub. No.: US 2006/0015138 A1
(43) Pub. Date: Jan. 19, 2006

(54) Emboli diverting devices created by microfabricated means

(76) Inventor: Michael Gertner, Menlo Park, CA (US)

Correspondence Address: Michael Gertner, PO BOX P, Menlo Park, CA, 94026, US

(21) Appl. No.: 11/184,276

(22) Filed: Jul. 18, 2005

Related U.S. Application Data
(60) Provisional application No. 60/589,131, filed on Jul. 19, 2004.
(60) Provisional application No. 60/645,682, filed on Jan. 21, 2005.

(51) Int. Cl. A61M 29/00

(52) U.S. Cl. 606/200

(57) ABSTRACT

A medical device for interposition between a first flow path and at least one second flow path is provided. The device includes a first surface facing toward the opening of at least one second flow path; and a second surface facing away from the opening of at least one second flow path. When the device is in the operative position, it extends less than the complete circumference of the first flow path and substantially covers the opening of at least one second flow path. The device contains one or more surface features to facilitate chronic implantation. The device further has one or more characteristic porosities. Different configurations are indicated depending on the pathophysiology being treated and dictate the characteristic porosity of the device. In some, cases blood is prevented from reaching the second flow path and in other cases, particulates traveling within the blood are prevented from reaching the second flow path. Methods of preventing emboli or blood flow into the second flow path are also provided. Methods and devices for delivery are also provided.

### PRIORITY INFORMATION

The current application claims priority to provisional patent application 60/589,131 filed on Jul. 19, 2004 and to provisional patent application 60/645,682 filed on Jan. 21, 2005.

### FIELD OF THE INVENTION

The present invention relates to implantable medical devices for filtering and/or diverting embolic material from blood. Also disclosed are methods employing the devices. More particularly, the invention relates to an implantable medical device and corresponding method for filtering or diverting embolic material in blood flowing at the branch of a major blood vessel.

### BACKGROUND OF THE INVENTION

Stroke is a leading cause of disability, death, and health care expenditure. In the United States 700,000 strokes, responsible for 165,000 deaths, occur each year (Ingall, T. (2004) J Insur Med. 36(2):143-52). It is the second most common cause of death worldwide, exceeded only by heart disease, and is the third most common cause of death in the U.S., as described in Heart And Stroke Statistical Update, Dallas, Tex., American Heart Association, 2000.

The greatest burden of stroke, apart from death, is serious long-term physical and mental disability. The treatment of stroke is associated with extremely high costs, with stroke-related illnesses responsible for greater than $49 billion in the U.S. in 2002 (Mancia, G. (2004) Clin Ther 26(5):631-48). Despite intensive research efforts, few effective treatments are available once stroke has occurred; thus, stroke prevention is a primary focus for health care providers.

A major portion of blood supplied to the brain hemispheres is by two major arteries in the neck, referred to as common carotid arteries (CCA), each of which bifurcates into an internal carotid artery (ICA) and, external carotid artery (ECA). Blood to the posterior portion of the brain is supplied by the two vertebral arteries.

Stroke is caused either by ischemia-infarction or intracranial hemorrhage. Infarction constitutes 85 to 90 percent of the total group in western countries (Sacco, R. L., et al. (1998), Classification Of Ischemic Stroke, Stroke: Pathophysiology, Diagnosis And Management, editors: Barnett, H. J. M., et al., third edition, Churchill Livingstone, N.Y., 271-83). The pathogenesis of ischemic stroke is complex with multiple potential mechanisms. Carotid plaque is one source of stroke, accounting for about 15-20% of cases (Petty, G. W., et al. (1999) Ischemic Stroke Subtypes, A Population-based Study Of Incidence And Risk Factors, Stroke, 30;2513-16). More frequently, infarcts are caused by more proximal sources of emboli, such as the heart and the aortic arch. One of the most common causes of cardioembolic stroke is nonrheumatic (often called nonvalvular) atrial fibrillation, prosthetic valves, rheumatic heart disease (RHD), congestive heart failure, and ischemic cardiomyopathy.

A recent population-based study found that the main identifiable subtype of ischemic stroke was cardioembolic in origin, at nearly 30% of cases, while all cervical and intracranial atherosclerosis altogether constituted about 16%. Further, multiple mechanisms often coexist (Caplan, L. R., (2000) Multiple Potential Risks For Stroke, JAMA, 283; 1479-80). Wilson, R. G. and Jamieson, D. G., in Coexistence Of Cardiac And Aortic Sources Of Embolization And High-grade Stenosis And Occlusion Of The Internal Carotid Artery, J. Stroke Cerebrovasc Dis., 9; 27-30, reviewed the experience of Petty et al. with patients who had high grade internal carotid artery stenosis or occlusion, and also had cardiac and aortic evaluation. Potential cardiac or aortic sources of emboli were present in 54% of patients; aortic arch plaques greater than 4 mm in diameter were found in 26% of patients with severe internal carotid artery occlusive disease.

In a seminal work, which retrospectively evaluated the causes of stroke in patients with unidentified causes (Amarenco et al. N. Engl. J. Med. 331: 1474-1479), 28.2 percent of patients who did not have an identifiable cause of the stroke were found to have aortic plaques that were greater than 4 mm in thickness. Prevention is possibly the most cost-effective approach to decreasing the burden of stroke. Available strategies to prevent stroke include medical treatment, surgery, and carotid stenting.

Current medical treatments include antiplatelet drugs, such as aspirin, ticlopidine, clopidogrel, and dipyridamol, for presumed atheroembolic and cardioembolic embolic origin. These treatments reduce the risk for a recurrent ischemic event by no more than 15-20%. Anticoagulants, such as warfarin, indicated for atrial fibrillation, reduce the risk by 60%; however, even in carefully conducted and monitored clinical trials, a substantial (25%) number of patients stopped anticoagulation due to side effects (Hart, R. G., et al. (1999) Antithrombotic Therapy To Prevent Stroke In Patients With Atrial Fibrillation: A Meta-analysis, Ann Intern Med., 131; 491-501). Furthermore, at least 10% of patients who would benefit from anticoagulant therapy for known proximal sources of emboli, cannot take anticoagulation due to the risk of falling, GI hemorrhage, etc.

Carotid endarterectomy was shown to be beneficial in medium and high grade symptomatic as well as in asymptomatic carotid stenosis, with a greater than 60% reduction in stroke rates (Chassin, M. R. (1998) Appropriate Use Of Carotid Endarterectomy, N. Engl. J. Med. 339, 1468-71). Nevertheless, a high proportion of recurrent stroke was unrelated to well-defined athero-thrombotic and embolic disease in the carotid artery, but to other causes including cardioembolism and probably aortic arch atheroembolism (Barnett, H. J. M., et al. (2000) Causes And Severity Of Ischemic Stroke In Patients With Internal Carotid Artery Stenosis, J. Amer. Med. Assoc. 283; 1429-36). In fact, strokes related to cardioembolism tended to be more severe. The population of patients with carotid stenosis often includes patients with severe cardiac disease, concomitant protruding aortic arch atheroma, atrial fibrillation, or congestive heart failure. The proportion of patients with such concomitant disease increases substantially in an elderly population. Thus, the risk of recurrent cardioembolic stroke, even in patients operated for carotid stenosis or given the anticoagulant coumadin, is estimated to be substantially higher. (Barnett, H. J. M., et al. (2000) Causes And Severity Of Ischemic Stroke In Patients With Internal Carotid Artery Stenosis, J. Amer. Med. Assoc. 283; 1429-36).

Carotid artery stenting has potential advantages of offering treatment to high risk patients with carotid stenosis, lowering peri-procedural risk, decreasing costs, and reducing patient inconvenience and discomfort. Preliminary results from clinical trials comparing carotid stenting to carotid endarterectomy have shown similar results, as described in Major Ongoing Stroke Trials (2000) Stroke 31; 557-2.

Manufacturing braided stents and prostheses is known in the art. For example, in the disclosures of U.S. Pat. No. 6,083,257, U.S. Pat. No. 5,718,159, U.S. Pat. No. 5,899,935, and, U.S. Pat. No. 6,494,907, the teachings of which are incorporated by reference as if fully set forth herein, there are described methods of manufacturing braided stents. Such braided stents present various advantages. However, they are generally made for the purpose of preventing stenosis and for supporting blood vessels. The relatively large mesh sizes employed, and the thickness and shape of the stent struts, make them less efficacious for filtering embolic material. Furthermore, stents are constructed to apply a radial force to the vessel in which they are implanted such that the frictional force generated by the radial force which keeps the stent positioned in the vessel.

Despite the above methods to treat and prevent stroke, 40-60% of patients who have strokes, have “cryptogenic” strokes. In a substantial number of cases, such strokes are thought to be caused by atherosclerotic debris from the aorta (Amerenco et al. (1994) N. Engl. J. Med., 331:1474-1479; Kallikazaros et al. (2000) Circulation 102: 265-268; and Bang et al. (2003) Ann. Neurology 54: 227-234). The best anticoagulant will not be effective for such cryptogenic strokes because the nature of the particulate matter is atherosclerotic in nature and not a clot.

The approach to prevention of such a multi-factorial and complex syndrome as stroke is necessarily multifaceted. Carotid angioplasty in combination with stenting, by itself, does not address additional sources of emboli (i.e. proximal sources), even after successful reduction of local stenosis. More efficient endovascular approaches prevent stroke need to take into account the complexity of cerebrovascular disease. In this context, an intravascular implant that also addresses prevention of emboli from proximal sources without regard to cause, can be a valuable addition to the arsenal of the practicing physician.

Introducing filtering means into blood vessels, particularly into veins, has been known for some time. However, filtering devices known in the art are designed for filtering blood flowing in the vena cava, and for stopping embolic material having a diameter of the order of centimeters. However, they are unsuitable to deal with arterial embolic material, with which the present invention is concerned, especially in cases where the dimensions of such material is typically on the order of microns. Furthermore, the flow of blood in the veins does not resemble arterial flow by its hemodynamic properties. However, when considering the possible cerebral effects of even fine embolic material occluding an artery supplying blood to the brain, the consequences may cause irreversible brain damage, or may be fatal. Nonetheless, even vena cava filters would benefit if their pore sizes were decreased in size to allow for greater area for blood flow.

In light of the short period of time during which brain tissue can survive without blood supply, there is significant importance to providing suitable means for preventing even small-sized embolic material from entering the cerebral circulation, so as to prevent brain damage, death, or diseases such as the slow onset of vascular dementia.

The size and shape of the struts that make up the filter device, the surface chemistry, the unique design for attachment to the blood vessels, and the porosity index thereof are features of the deflecting device of the present invention, as explained below. By contrast, in venous blood filters currently known in the art, particular attention has not been given to the size of the filaments. In a typical vena cava filter, the goal of the filter is to prevent large (i.e. greater than 1-5 mm) pieces of material from reaching the lungs which results in a pulmonary embolus. The bar is much higher when dealing with brain tissue where the size of emboli which can cause a problem is on the order of 100-200 microns. It is noted that embolic material in venous blood is made up only of blood clots, while in arterial blood, it is necessary to deal with emboli featuring different materials and combinations of materials, such as blood clots and atherosclerotic plaque debris, etc.

Thus, filtering devices known in the art are generally of a complex design, which renders such devices unsuitable for implantation within carotid arteries, and unsuitable for handling fine embolic material. However, when considering the possible cerebral effects of even fine embolic material occluding an artery supplying blood to the brain, the consequences may be fatal or may cause irreversible brain damage. Therefore, it is of significant importance to provide suitable means for preventing small embolic material from entering the proximal cerebral vessels. The present invention is designed to meet these needs.

### SUMMARY OF THE INVENTION

Accordingly, one aspect of the invention provides a medical device adapted for interposition between a first flow path and at least one second flow path. The device includes a first surface facing toward an opening of the at least one second flow path, and a second surface facing away from the opening of the second flow path. When the device is in operative position it extends less than the complete circumference of the first flow path and substantially covers the opening of the at least one second flow path. At least one portion of the first surface or second surface is adapted for chronic implantation of the device.

In one embodiment of the invention the device is substantially planar.

In another embodiment, at least one portion of the device includes one or more flanges for securing the device to the first flow path or at least one second flow path. One or more flanges are attached to the first surface for engaging one or more walls of the at least one second flow path. Preferably, the device includes one or more flanges attached to the second surface for engaging a portion of the wall of the first flow path.

In certain embodiments, the device further includes an outer portion which extends beyond the opening to the at least one second flow path to contact a wall of the first flow path, and a second portion which substantially covers the opening of the second flow path. In these embodiments, the device can also include the attachment of a flange to the first or second portion.

In other embodiments, at least a portion of the device has an undeployed, unexpanded configuration and a deployed, expandable configuration.

In more specific embodiments, the device has a specified thickness. For example, a thickness may be defined between the first surface and the second surface of less than about 100 microns. The thickness between the first surface and the second surface may be less than about 25 microns. Preferably, the thickness between the first surface and the second surface is less than about 5 microns.

At least a portion of the device may be porous. The porous portion of the device may include a plurality of struts which define a plurality of device openings and which are characterized by a cross-sectional shape and a largest cross-sectional dimension. The largest cross-sectional dimension may be less than 50 microns, and in certain embodiments is between 0.5 and 20 microns. The porosity index of the porous portion of the device can be 70-80%, from 80-90%, or from 90-95%, or even up to 99%.

In one embodiment, the cross-sectional shape is configured to minimize the frictional drag of fluid traveling over said strut. The cross-sectional shape may be shaped like an airplane wing to reduce drag.

In some embodiments, the device is a component of a larger device such as a stent.

The plurality of struts may comprise a coating.

In preferred embodiments, the first flow path is the aorta of a patient, and the second flow path transmits blood to the cerebral circulation.

The device includes, or is made of, a biomaterial such as acrylics, vinyls, nylons, polyurethanes, polycarbonates, polyamides, polysulfones, poly(ethylene terephthalate), polylactic acid, polyglycolic acid, polydimethylsiloxanes, and polyetheretherketones, metals, metal alloys, ceramics, glass, silica, and/or sapphire. The acrylics may be selected from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; the vinyls are selected from ethylene, propylene, styrene, vinyl chloride, vinyl acetate, vinyl pyrrolidone, and vinylidene difluoride; the nylons are selected from polycaprolactam, polylauryl lactam, polyhexamethylene adipamide, and polyhexamethylene dodecanediamide; the metals and metal alloys are selected from titanium, stainless steel, cobalt chromium, gold, silver, copper, and platinum and their alloys; and the ceramics are selected from silicon nitride, silicon carbide, zirconia, and alumina, including combinations of such biomaterials. The biocompatible material is preferably selected from the group consisting of stainless steel, nickel-titanium, titanium, silicon and cobalt-chromium.

In one embodiment of the invention, the device includes one or more similar or different coatings disposed on at least one or more portions of the first and/or second surfaces. The coating may be adapted for tissue ingrowth.

The outer portion includes, in another embodiment, a structure which irreversibly conforms to the contour of the first flow path. The outer portion may include an energy activateable coating.

The device includes, in one embodiment, a coating adapted for adhesion upon chronic contact with the first flow path. The coating on at least one portion of the device is relatively hydrophobic.

Alternatively, at least one portion of one coating comprises a bioactive agent. The bioactive agent may be one or more of thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti-secretory agents, actin inhibitors, remodeling inhibitors, antisense nucleotides, anti-metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, anti polymerases, antiviral agents, photodynamic therapy agents, gene therapy agents, and/or scar inducing agents. The bioactive agent may be selected from the group consisting of attached active groups, a radioactive material, gene vectors, and medicaments.

Another aspect of the invention includes a method of manufacturing a device for interposition between a first flow path and at least one second flow path. The method includes patterning a substantially planar substrate to form a plurality of struts which define a plurality of porous cavities which extend through the substrate.

The patterning may include chemical or electrochemical etching.

At least one step of the manufacturing process of the device may include a self-assembly process. The patterning preferably includes lithography.

In certain embodiments of the method, at least one portion is manufactured using a mold with features smaller than one micron.

The patterning alternately includes physical vapor deposition. The lithography may include nanoimprint lithography.

Yet another aspect of the invention includes a device adapted for interposition between one first flow path and at least one second flow path. The device includes a first surface facing toward an opening of the at least one second flow path, and a second surface facing away from the opening of the second flow path. One portion of the device is adapted to reversibly admit a catheter therethrough.

The invention includes in still another aspect, a wire that includes a proximal end and a distal end, the proximal end adapted for extracorporeal manipulation, the distal end adapted for intravascular manipulation, the distal end further comprising a grasper mechanism adapted to hold a device in an undeployed state. The grasper can release the device through a mechanism initiated by an operator at the proximal end of the wire.

The grasper mechanism preferably includes an electromagnet. The grasper mechanism may also include a heat releasable polymer weld between the wire and the device. Alternatively, the grasper mechanism includes a metallic bond between the wire and the device.

The grasper mechanism is, in one embodiment of the invention, a mechanically actuateable pair of claws.

Another aspect of the invention includes a catheter assembly which includes a catheter adapted for use in a vascular system of a subject, a device as described above within the catheter, and a wire as described above reversibly contracting the device. The catheter assembly may also include a second lumen adapted to transmit a distinct wire for guidance through the vascular system.

Yet another aspect of the invention includes a method of preventing emboli from reaching a second flow path from a first flow path in a patient. The method includes delivering a device in a substantially undeployed configuration to the intersection between a first flow path and at least one second flow path, deploying the device such that the deployed profile engages less than the full circumference of the first flow path, and allowing healing of the device such that the device remains in place on a chronic basis. The method also includes applying an energy source to a region of said device to attach the device to the first or to the at least one second flow path. In one embodiment of the invention, the first flow path is an aortic arch of a patient, and the at least one second flow path branch is the right brachiocephalic artery, the left common carotid artery and/or the left vertebral artery. The method may also include releasing a therapeutic agent from the device.

Another aspect of the invention includes a method of protecting a patient against embolization for a period of time longer than one operative procedure. The method includes percutaneously guiding a catheter to a target site at the interface between a first flow path and at least one second flow path; deploying a substantially planar device as described above at the target site and securing the device at the interface between the first and at least one second flow path. Preferably, the deploying step includes a disengaging step wherein the device is disengaged from a wire. In one embodiment of the invention, the disengaging step includes electrochemically degrading an attachment between the pusher wire and the device.

In certain embodiments of the invention, a sizing balloon is inserted into the second flow path and a correctly sized implant is chosen for implantation at the second flow path.

In other embodiments, the device is localized to the intersection region by visualizing a component of said device under fluoroscopy, CT, ultrasound, or MRI imaging.

These and other objects and features of the invention will be more fully appreciated when the following detailed description of the invention is read in conjunction with the accompanying drawings.

### BRIEF DESCRIPTION OF THE DRAWINGS

FIG. 1A is a front view of a medical device constructed in accordance with the present invention;

FIG. 1B is a perspective view of the medical device shown in FIG. 1A;

FIG. 1C is a front end-on view of the medical device shown in FIGS. 1A and 1B;

FIGS. 2A-2D illustrate perspective views of a medical device with anchoring flanges constructed in accordance with a preferred embodiment of the invention;

FIG. 3A is a perspective view of a medical device similar to that of FIG. 2A;

FIG. 3B is a perspective view of a medical device similar to that in FIG. 2B in which the anchoring flanges are folded inward in accordance with another embodiment of the invention for insertion into a flow path;

FIG. 3C illustrates a medical device constructed in accordance with the present invention in which the entire medical device is in a reduced state for insertion into a flow path;

FIG. 4 is an expanded illustration of a grid structure comprising the medical device in accordance with another preferred embodiment of the invention;

FIG. 5 is a front end-on view of an implanted medical device which is positioned according to yet another embodiment of the invention;

FIG. 6 shows multiple medical devices constructed and implanted in accordance with the present invention in which the medical devices have been inserted and positioned in a flow path;

FIGS. 7A illustrates one embodiment of a method for loading the device of the invention into the distal end of a catheter;

FIG. 7B illustrates one embodiment of a method for loading the catheter, with the device of the invention inside, into a sheath for introduction into a patient.

FIGS. 8A-8D illustrate steps in the placement of the device at the flow path inlet in accordance with one embodiment of the method of the invention;

FIG. 9A illustrates a front end-on view of a medical device having coatings on the first and second surfaces in accordance with an embodiment of the present invention;

FIG. 9B illustrates a longitudinal cross-section of the device of the present invention;

FIGS. 9C-D illustrate magnified strut cross-sections in accordance with the described invention;

FIG. 10A shows a representation of the fluidic flow across the struts of the current invention;

FIG. 10B shows various cross-sectional shapes of the struts in accordance with other embodiments of the current invention;

FIG. 11 illustrates a perspective view of a medical device constructed in accordance with the present invention which is configured to substantially cover more than one flow path inlet;

FIG. 12 shows a side view of a medical device similar to that of FIG. 11, which has been inserted and positioned over multiple flow paths according to another embodiment of the present invention; and

FIGS. 13A-B show a configuration of the device which enables access to the cerebral vasculature by a catheter.

It is to be understood that the foregoing drawing descriptions and the detailed description below are provided primarily for the purpose of facilitating the understanding of the conceptual aspects of the invention and various possible embodiments thereof. It is to be further understood that the embodiments described are for purposes of example and that the invention is capable of being embodied in other forms and applications than described herein.

### DETAILED DESCRIPTION OF THE INVENTION

Definitions

Unless otherwise indicated, all technical and scientific terms used herein have the same meaning as they would to one skilled in the art of the present invention. It is to be understood that this invention is not limited to the particular methodology and protocols, as these may vary.

The term “flow path” refers to a generally enclosed region which contains fluid flow. The “first flow path” refers to the proximal portion of the flow of blood or where the blood is flowing from. The “second flow path” refers to the distal portion or where the blood is flowing to (e.g. into a branch vessel).

An exemplary flow path is a region of the cardiovascular system such as the aorta, vena cava, carotid artery, iliac artery etc. In this example, a first flow path is the aorta and the second flow path is a carotid artery.

Flow paths are not limited to blood vessels. The chambers of the heart are also enclosed regions in which fluid flows. A first flow path may be the atrium and a second flow path is could be a ventricle. In a pathologic condition such as an atrial septal defect or a patent foramen ovale, a first flow path could be the right atrium and the second flow path may be the left atrium.

Additional exemplary flow paths are malformations such as aneurysms, or outpouchings of blood vessels. The walls of such malformations may comprise all three layers of the vessel wall (true aneurysm) or may be comprised of a fibrous “pseudocapsule” such as in a false aneurysm. The aneurysm may contain a clot without active blood flow such that the aneurysm was formerly a second flow path but does not contain active blood flow any longer. In any, it remains desirable to prevent transmission of pressure between the first flow path and the aneurysm sac to prevent rupture.

The term “filter” has its ordinary meaning and in addition refers broadly to devices, materials, and the like that are able to allow certain components of a mixture to pass through while retaining or deflecting other components; as used herein, it is a device which prevents material from entering a second flow path from a first flow path. Under this broad definition, a filter can retain or it can divert emboli. For example, a filter may comprise a mesh with pores sized to allow a blood product (e.g., plasma, protein, cells) to pass through, while retaining other components such as embolic material. The term “filter” is not limited to the means by which certain components are retained.

A “deflector”, falls into a category of filters, which filter by diverting material to a different place rather than filtering by storing the material in the device itself.

The terms “opening” and “pore” have their ordinary meaning and are also used interchangeably to refer to a continuous open channel or passageway from one surface of a filter to the other surface.

The term “porosity index” is generally defined as the ratio of open area for fluid flow to the total area of the area where fluid would flow if there was not a device present. The porosity index is generally expressed as a percentage.

The term “specific porosity index” refers to the porosity index of one pore or opening as a ratio of the pore (open) area to the material defining the opening. This term defines a minimum strut size around a pore or an opening. The specific porosity index is the same as the porosity index when the device is the same throughout and the porosity index is different from the specific porosity index when there are different sized pores throughout the device. A related term is the “average specific porosity index” which refers to the index of each pore as averaged over the surface of the filtering region 130 of the stent.

The terms “fluidic connection,” “fluidic contact,” and the like refer to the ability of a fluid component (e.g., blood) to flow from one element or flow path to another.

The term “blood” has its ordinary meaning and also refers to all formulations of the fluid and/or associated cellular elements and the like (such as erythrocytes, leukocytes, platelets, etc.) that pass through the body's circulatory system; blood includes, but is not limited to, platelet mixtures, serum, and plasma.

The terms “emboli,” “embolic material,” and the like refer broadly to any undesired or occluding material in vessels and other body passageways. Such material may include atheromas or atheromatous emboli, thrombi, or thromboemboli. An atheroma is a mass of plaque of degenerated, thickened arterial intima occurring in atherosclerosis. A thrombus is an aggregation of blood factors, primarily platelets and fibrin with entrapment of cellular elements, frequently causing vascular obstruction at the point of its formation. An embolus is a clot or other plug brought by the blood from another vessel and entering into a second one, thus obstructing the circulation, generally. “Atheroemboli” are emboli which are composed of atherosclerotic particles; “thromboemboli” are clot particles which can originate from many different sources including atherosclerotic plaques, fibrillating chambers of the heart, etc. Many emboli will be of mixed origin.

The term “substantially cover” refers to placing a medical device such that the opening of the flow path is covered to such a degree that detrimental amounts of the occluding agent are not able to migrate or flow into the “substantially covered flow path”. In certain embodiments, “substantially cover” refers to substantially covering one or more flow path inlets.

As used herein, the terms “implant” and “implanted” include devices that are implanted into the body. Temporarily implanted devices are those which are implanted acutely during one procedure such as, for example an operation, interventional procedure, or other procedure. When the procedure is finished, the device is removed from the patient. Chronically implanted devices are those devices which remain implanted after the procedure is finished and necessarily are configured for chronic implantation.

The term “branch” is distinguished from “bifurcation” in that a branch of a vessel is an entirely new vessel which is derived from the first flow path or first vessel (e.g., the carotid artery is a branch of the aorta) and typically has a different name. The branch has its own wall just after branching from the vessel of origin. Furthermore, the first vessel or flow path continues (with the same name) beyond the branch. In a bifurcation, a single vessels splits into two vessels with a common wall at the beginning of the bifurcation. For example, the common carotid artery branches into the external carotid and the internal carotid arteries; the iliac artery divides into the external and internal iliac arteries. The first vessel or flow path becomes (and is so named) an entirely new blood vessel.

As used herein, the terms “biocompatible” or “bioactive/biocompatible” will refer to a molecule or a continuum of atoms or molecules (e.g. a surface) having a desired and expected biological or chemical activity (e.g. cellular ingrowth, cellular repulsion, prevention of clotting, selection of specific cells such as endothelial cells).

The terms “patient,” “subject” and “individual” are used interchangeably herein to refer to any target of treatment. Any subject in which a flow path containing blood may be found may be treated with the devices and methods in accordance with the present invention. For example, canine, feline, equine, bovine, and porcine hosts are preferred subjects. More preferably the subject is a human.

The terms “protein” , “polypeptide” or “peptide”, as used herein, refer interchangeably to a biopolymer composed of amino acid or amino acid analog subunits, typically some or all of the 20 common L-amino acids found in biological proteins, linked by peptide intersubunit linkages, or other intersubunit linkages.

The term “controlled release” is intended to refer to any bioactive material containing formulation in which the manner and profile of drug release from the formulation are controlled. The term “controlled release” refers to immediate as well as non-immediate release formulations, with non-immediate release formulations including but not limited to sustained release and delayed release formulations.

The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time and thus may or may not be “sustained release.”

A “therapeutic treatment” is a treatment administered to a subject who displays symptoms or signs of pathology, disease, or a disorder, in which treatment is administered to the subject for the purpose of diminishing or eliminating those signs or symptoms of pathology, disease, or disorder.

A “preventative treatment” is a treatment administered to a subject which is intended to prevent the occurrence of a pathology. For example, coumadin is a drug which prevents clot formation. When given to a patient with a disease such as atrial fibrillation, it is intended to prevent the formation of stroke causing emboli.

The terms “nucleic acid molecule” or “oligonucleotide” or grammatical equivalents herein, refer to at least two nucleotides covalently linked together, and typically refers to RNA, DNA and cDNA molecules. A nucleic acid of the present invention is preferably single-stranded or double-stranded, and will generally contain phosphodiester bonds, although in some cases nucleic acid analogs are included that may have alternate backbones comprising, for example, phosphoramide, phosphorothioate, phosphorodithioate, and/or O-methylphosphoroamidite linkages.

As used herein, “effective amount” or “pharmaceutically effective amount” of an active agent refers to an amount sufficient to derive a measurable change in a physiological parameter of the target or patient and/or to provide or modulate active agent expression or activity through administration of one or more of the pharmaceutical dosage units. Such an effective amount may vary from person to person depending on their condition, height, weight, age, and/or health, the mode of administering the active agent, the particular active agent administered, and other factors. As a result, it may be useful to empirically determine an effective amount for a particular patient under a particular set of circumstances.

All publications and patents cited herein are expressly incorporated by reference for the purpose of describing and disclosing the devices and methodologies that might be used in connection with the invention.

Device of the Invention

The invention includes, in one aspect, a device adapted for placement at the interface of a first and second flow path of a patient and further adapted to retain emboli in a first flow path by a deflection mechanism and prevent their introduction into a second flow path. The device is further adapted to create minimal resistance to blood flowing from the first flow path to the second flow path. It has been discovered that a medical device having the proper thickness and strut dimensions and porosity indices and specific indices, as described herein; and which has been constructed to fit over a single flow path in a patient, provides a number of beneficial biological uses. Considered below are the devices of the invention and methods utilizing the devices.

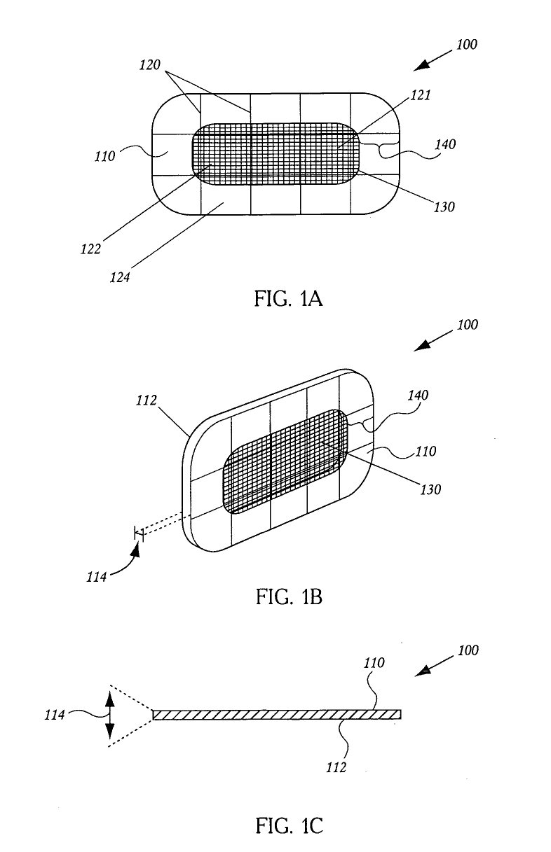

Referring to FIGS. 1A-1C, the implantable device 100, or filtering device, described below is composed of a first surface 110 and second surface 112. A thickness 114 is defined between surfaces 110 and 112. When properly positioned in a patient, the device substantially covers the opening, or interface, of one or more second flow paths which are in fluid communication with a first flow path, as described below. The first surface 110 is the surface facing the second flow path and the second surface 112 is the surface facing the first flow path. Device 100 is preferably adapted to be disposed in a vessel (e.g. artery) to allow passage of fluid (e.g. blood) to the second flow path with minimal resistance and to therefore deflect emboli in the blood away from the second flow path, generally preventing their entrance into the second flow path. Flow is generally in the direction of the first flow path to the second flow path.

In some embodiments Device 100 is a sub-component of a larger device. For example, device 100 can be a component of a stent. The stent can be used to divert emboli from one flow path to a second flow path. In this embodiment, device 100 forms the critical (filtering or diverting) part of the diverter device and the stent holds device 100 in place. Device 100 can be manufactured separately from the stent and then welded to the stent or device 100 can represent further processing of the stent after the stent has been manufactured.

Device 100 may be further comprised of one or more regions. In a preferred embodiment, as illustrated in FIGS. 1A-1C, the device is comprised of two regions, 130 and 140. Region 130 is the region which substantially covers the opening of the second flow path when the device is in its operative position. Region 130 can be adapted to cover many types of flow openings; for example, it can be adapted for placement over the opening to an aneurysm, over a septal defect in a wall of the heart, over a defunct portion of the heart such as an atrial appendage or aneurysm in a wall of a ventricle. Other adaptations for region 130 include bifurcations of blood vessels such as the external and internal carotid and the internal and external iliac blood vessels. Further adaptations include use in the venous system such as in the vena cava for prevention of pulmonary emboli. In some preferred embodiments, region 130 is adapted for placement at branch vessels such as the carotid, inominate and renal branches off the aorta.

Region 140 has a structure adapted to optimize the position of the device and to secure it in the region of the second flow path, preferably on a chronic and ongoing basis. Region 140, in some embodiments is substantially planar, and in other embodiments, is tubular or is configured as a stent. In some embodiments, region 140 includes attachment members such as flanges. Regions 140 and 130 can be attached to each other by any of a number of methods known to those skilled in the art such as welding, laser welding, soldering, epoxy based methods, etc.

Region 130 can have a porosity, porosity index or specific porosity index which may, but does not have to be the same as region 140. Alternatively, regions 130 and 140 can have the same porosity, porosity index or specific porosity index. The degree of porosity can be chosen with respect to the medical condition being addressed.

In one embodiment, porosity of region 140 is optimized for tissue ingrowth; in this case, the porosity between struts 120 can be 10-50 microns which, in many cases is optimal for tissue ingrowth, being approximately the same size as an endothelial cell. In other cases, the porosity is greater than 150 microns, such as from 150 to 500 microns, or even as great as 1 mm. Although tissue ingrowth may not be as great or rapid, the overall device will contain less material and be lighter and possibly easier to implant when the pore size of region 140 is larger.

In some embodiments, a minimum distance between any two struts of the filtering region 130 or non-filtering portion 140 is defined. In other embodiments, a maximum distance between any two struts of the filtering region 130 or non-filtering portion 140 is defined. Both these maximum and minimum distances are for struts that run substantially parallel. For example, in some embodiments, the minimum distance between two adjacent parallel struts is at least 50 microns while in some embodiments, the minimum distance between adjacent parallel struts is at least 75 microns. In still other embodiments, the minimum distance between the struts is at least 100 microns. Such embodiments are useful for diverting or filtering emboli when it is crucial to maintain the flow of blood and its components. In some embodiments, the maximum distance between adjacent struts is 100 microns and in other embodiments, the maximum distance between adjacent struts is 50 microns. Such embodiments are beneficial for certain types of tissue adhesion and ingrowth or in the case when it is desirable to prevent both emboli and flow from reaching a second flow path such as, for example, when treating an aneurysm.

In some embodiments, region 130 is adapted to baffle the flow of fluid at or near its surface. A baffle has its ordinary meaning and in addition as used herein, a baffle refers to a structure with specific features to direct flow in a given direction. Such features can in some embodiments enhance the diverting effects of the filtering surface 130. For example, filtering region 130, rather than having a flat profile can be at least partially hemispherical or elliptical. In such an adaptation, when fluid flows over the profile, embolic material in the blood will be pushed to the outer flow lamina because due to centrifugal forces.

In the embodiments where high flow occurs through region 130, for example, in an embodiment which includes blood vessels which travel to the cerebral circulation, the porosity, porosity index, or specific porosity index are adapted to prevent emboli from reaching the second flow path; with chronic high flow through the device, tissue ingrowth is unlikely to occur within the filtering portion of the device 130. Portion 130 of the device will therefore remain patent.

In some embodiments, region 140 is optimized for securing the device to the first flow path. Region 140 may be made from any number of suitable materials, and have a number of configurations. In one embodiment of the invention region 140 is formed from a solid, impervious material. Preferably, as shown in FIGS. 1A-C, region 140 comprises one or more pores 124. Pores 124 may be created by any suitable means (described below), such as being defined by struts 120. Alternatively, pores 124 may be created by drilling or etching, e.g., laser drilling or etching.

Region 140 may contain pores optimized for endothelial or fibrous ingrowth. In some embodiments, region 140 contains a photo- or heat activateable coating material activated when region 140 is placed in contact with the wall of the first flow path. In some embodiments, region 140 can have a coating, a coating and a drug, or can have a drug attached without an involved coating. The drug can have multiple functions; for example, it can encourage in-growth into portion 140 or it can prevent ingrowth into region 140. In certain embodiments, region 140 contains an antibody bound to the surface which attracts endothelial progenitor cells and hastens the endothelialization process. Such antibodies are known in the art and have been described, e.g., in U.S. Pat. No. 6,726,923, issued April 27, which is incorporated by reference herein.

Regions 140 and 130 can be formed from the same or different materials. Preferably, region 140 is more flexible and/or more ductile than the portion 130. Such enhanced ductility and flexibility allows for portion 140 to be urged against the wall of the first flow path and be plastically deformed in order to be secured to the wall of the first flow path. The ductility of the device will allow it to conform to a rough and uneven surface, e.g., of an atherosclerotic vessel.

In another embodiment, struts 121 define a porosity 122, porosity index, or specific porosity index along the flow path such that device 100 is made substantially impervious to the passage of blood and any of its components from the first flow path to the second flow path. It is the combination of decreased porosity and low blood flow which leads to substantial impermeability. That is, the higher the blood flow, the smaller the pores need to be in order to prevent blood from flowing through the device. The porosity, or impermeability of the filtering device, is selected in order to reduce the pressure on, for example, an aneurysm, or to close a structural defect in an organ such as the wall of the heart (e.g. a patent foramen ovale). Such a porous structure, which is substantially impermeable to flow, will ultimately encourage fibrous ingrowth, i.e. endothelialization, forming a permanent barrier between the first flow path and the second flow path which would be desirable in a disease state such as a patent foramen ovale or an aneurysm.

An exemplary shape of the device is illustrated in FIG. 1. Device 100 is formed from a plurality of first struts 120 and second struts 121 which define a plurality of device openings (the porosity) 122 and 124 and by definition the porosity index and specific porosity index. Alternatively, device 100 is formed of a single region 130, and comprises struts 121 which define a plurality of device openings (the porosity) 122. The device, as viewed from the front, may be any number of shapes. For example, the device may be rectangular, square, oval, circular, or a combination of these shapes. As noted above, in one embodiment, the device functions to filter by deflection, emboli from blood flowing at the interface of a first and one or more second flow paths of a patient. Thus, the device should have a shape such that once it has been positioned in the patient, it substantially covers the second flow path. Exemplary horizontal and vertical dimensions of the device are 0.25 to 1.0 cm for arterial branches such as the vertebral arteries, 0.75 to 1.5 cm for arteries such as the carotid or renal arteries, 1.0 to 2.0 cm for arteries such as the inominate, subclavian, or brachiocepahlic arteries, and 0.5 to 2.0 cm for defects such as a patent foramen ovale or atrial septal ostium secundum defect.

In some embodiments, struts 120 and/or 121 have a cross-section characterized by a height and a width. In these embodiments, the height of struts 120 define thickness 114. Thickness 114, according to one embodiment of the invention, is less than about 50 microns. Preferably the thickness 114 is less than about 25 microns and more preferably, thickness 114 is less than 10 microns. Thus, the height of struts 120 is preferably less than 50 microns, and more preferably, less than about 25 microns, and even more preferably, less than about 10 microns. In some embodiments, the strut width is approximately the same size as the strut height although it may be desired or required (by the manufacturing process) that the width:height (aspect ratio) ratio be from 0.2 to 5.

In a preferred embodiment of the current invention, the cross-section of the struts is substantially circular or ovoid in which case a diameter or largest diameter characterizes the thickness of the strut and of the device 114. In such an embodiment, a preferred diameter is less than 20 microns and preferably less than 5 microns. Further in such an embodiment, thickness 114 is the diameter of the cross-section of the strut. In other embodiments, the cross-sectional profile of the struts is not simply characterized by a known shape, in which case height and width or a diameter would be the correct characterization. In some embodiments, the “largest cross-sectional dimension” is used to characterize the thickness of the cross section and hence the thickness 114 of the device.

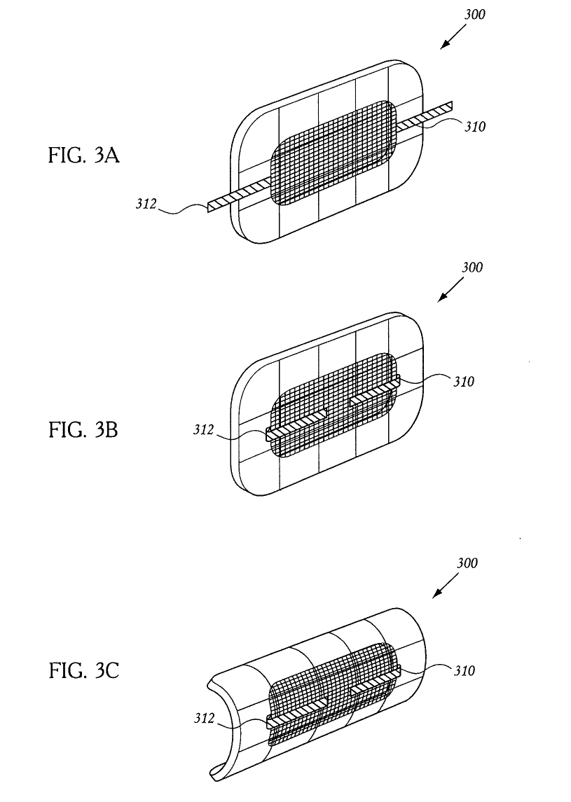

In certain embodiments of the invention, a portion or substantially all of the device, is made of a material having an elasticity suitable for expanding from a first contracted, or undeployed configuration (in which it is delivered to the flow paths of interest), to a second uncontracted, and deployed configuration. Expansion from one configuration to a second configuration is accomplished by means which will be further described below and with reference to FIGS. 3A-3C.

Suitable device materials include biocompatible materials such as acrylics, vinyls, nylons, polyurethanes, polycarbonates, polyamides, polysulfones, poly(ethylene terephthalate), polylactic acid, polyglycolic acid, polydimethylsiloxanes, and polyetheretherketones, metals, ceramics, glass, silica, and sapphire. Suitable acrylics include methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide. Suitable vinyls include ethylene, propylene, polypropylene, styrene, polystyrene, vinyl chloride, vinyl acetate, vinyl pyrrolidone, and vinylidene difluoride. Preferred nylons include polycaprolactam, polylauryl lactam, polyhexamethylene adipamide, and polyhexamethylene dodecanediamide. Preferred metals are titanium, nickel titanium, stainless steel, cobalt chromium, gold, silver, copper, and platinum and their alloys. Suitable ceramics include silicons such as silicon nitride and silicon carbide, zirconia, and alumina. Combinations of such biomaterials may also be useful in the present invention.

Materials of particular interest are stainless steel, cobalt-chromium, titanium, and nickel-titanium. It is important to realize that materials possess remarkably different properties depending on the size of the cross-section and their shape. For example, stainless steel with a thickness greater than a 1-2 mm and length smaller than 1 cm has a stiff bending modulus; however, when the thickness is 10-20 microns at the same length, the steel is highly flexible. The similar case exists with nitinol, cobalt-chromium, and most other materials.

In the embodiments where it is desirable to promote endothelial overgrowth, it may be particularly advantageous for the material to be a metal; certain metals (e.g. cobalt chrome, stainless steel, and nickel-titanium), when configured into a porous shape, are known in the art to promote healing and endothelialization.

For the most part, the porosity index and placement of the device in the patient depend on the medical disease to be treated by implantation of the device. Treatment of many of the diseases potentially treatable with the device of the current invention are well-known to persons skilled in the art and will not be described herein for the sake of brevity.

In a preferred embodiment, the first flow path is the aortic arch of a patient, and the second flow path is the right brachiocephalic artery, the right carotid artery, the left common carotid artery, the right vertebral artery, or the left vertebral artery.

Flanges

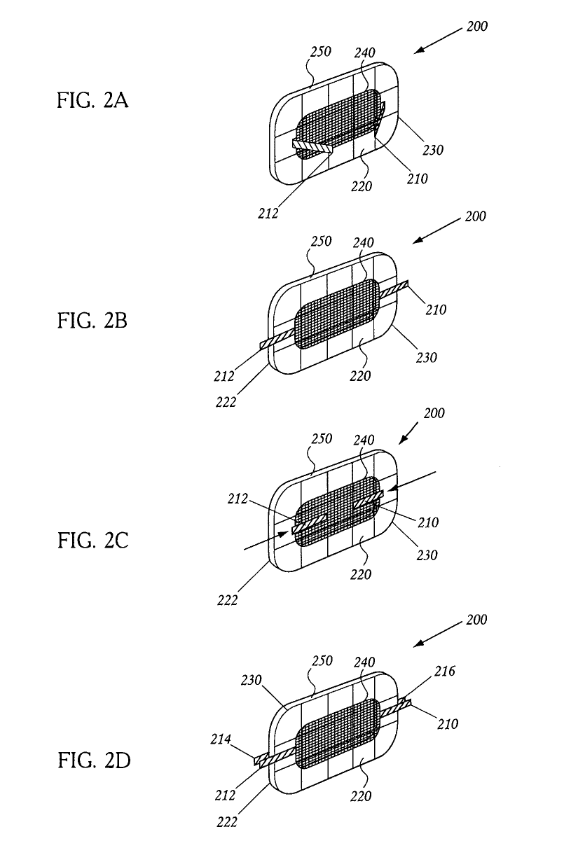

Another component of the device comprises structures to secure it to the second flow path for chronic implantation. As shown in FIGS. 2A-2C, device 200 includes one or more flanges 210 and 212 attached to inner portion 240 of first surface 220. The term “flange” is meant to encompass the structure that extends outward, and preferably perpendicular, to the planar surface 240 when the device is in the operative position and in addition, causes a frictional force between it and the wall of the second flow path. When the device is not in the operative position and not in a compressed and folded position (see below), flanges 210 and 212 project laterally or substantially laterally from the surface(s) of the device (FIG. 2B) due to a spring effect of the flanges.

The flanges may be attached to the surface of the device by a number of methods, e.g., flexible metal or polymeric weld. When force is applied in the direction of the arrows in FIG. 2C, the flanges are biased inward toward one another. Such is the case when the device is in its compressed and undeployed state. When the force is released (deployment), the flanges expand outward away from the surface; such is the case when the device is deployed and is in an uncompressed state. A flange provides distributed contact (i.e. the force of contact is spread over an area) at the wall of the second flow path wall and exerts pressure against the wall to hold or retain device 200 in place by frictional forces. Frictional forces function to hold the device in place just after deployment and then chronically with endothelial overgrowth in the longer term.

FIG. 2B depicts the flanges in the fully expanded state pointing away from surface 240 (this is the equilibrium position of the flanges) when the device in not within a vessel. FIG. 2C depicts the flanges in their fully undeployed state, pointing inward and flush with surface 220. In this case, force in the direction of the arrow to maintain the flanges in the compressed, undeployed state. When implanted in a flow path, the flanges attain a position between the deployed and undeployed states such that the flanges apply force to the walls of the second flow path and therefore hold the device in place (see below). In many embodiments, the direction of the flanges in their operative position is generally perpendicular to the surface of the device 220.

Optionally, as illustrated in FIG. 2D, an additional flange 214 and/or flange 216 may be attached to the first surface 222 which faces and secures the device to the first flow path. When device 200 is positioned within the patient in the operative position, flanges 210 and 212, and optionally flanges 214 and 216, engage the walls of the second and first flow paths respectively, thereby securing device 200 against the opening of the second flow path (see below). In certain embodiments, flanges 210 and 212 are the only flanges required on the device, as illustrated in FIG. 2B. In such an embodiment, flanges 210, 212 engage the walls of the second flow path and the device is further urged by the operator into a position where the device is held tightly against the walls of the first flow path after implantation and by outer region 220. Migration of the device is prevented by the arterial blood pressure along portion 240 of the device (and ultimately cellular ingrowth) as well as the flanges anchored into the second flow path and optionally, the first flow path.

Flanges 210, 212, are shown in FIGS. 2A-2C as being attached to the outer edge of inner region 240 of device 200. It is to be understood, however, that one or more flanges may be attached anywhere on device 200, such as device edge 250. The flanges will generally extend substantially parallel and toward the center of device 200 when in the compressed, undeployed position; the device can be further biased to press against the flow path walls when in the operative, deployed position. For example, when the device is positioned in a patient, flanges 210 and 212 will be biased to press against the wall of the second flow path and, optionally, flanges 214 and 216 will be biased to press against the first flow path. This mutual biasing of flanges contributes to anchoring of device 200 to the interface of the first and second flow paths.

The flanges themselves may optionally be turned or curved toward the wall of the second flow path. This curvature of the flange may bias the distal portion of the flange to press against and embed into tissue at a region of the first or second flow path once inserted into a patient. The insertion into the wall of the flow path has the advantage of substantially preventing dislodgement of the device.

In some embodiments, composite materials can be produced to achieve the desired designs and constraints of the present invention. For example, flanges 210 and 212 can be attached (e.g., welded) to the device after the filter portion is manufactured (220 and 240). Planar portions of the device 220 and 240 can be manufactured from a material which is optimal for the geometric constraints of the filter; the flanges can be produced from the same or substantially different material to achieve its intended configuration. An exemplary device has flanges formed from metal, such as stainless steel, with a thickened claw at the distal end and with a tapered portion close to the weld at the filter portion 240. The claw facilitates anchoring into the wall of the second flow path and the tapering portion facilitates flexibility for self-expansion during deployment. Although the strength and rigidity of the flange and its attachment to the device may be important in the short term (i.e. less than 7 days), over a longer period of time, the rigidity of the attachment is less important because the device, once inserted into the patient, will heal into the vessel wall.

An important feature of the flange aspect is that the tip, or end, of the flange or claw not apply an undue amount of pressure in one spot along the wall of the second flow path. In certain embodiments, the flanges are rounded at their distal end to render them atraumatic and to allow them to spread out their to the vessel wall over a large portion of the wall of the vessel. Optionally, or additionally, flanges may be covered with soft material coverings and/or provided with an atraumatic bulb, ball, mesh, or cylindrical tip. In one embodiment, the soft covering is a hydrogel coating which will absorb liquid when it contacts the vessel wall, thereby expanding to a final configuration which renders the flange atraumatic to the wall.

Optional markers (not shown), such as radio opaque markers, may be attached to any part of the device to aid the physician in the proper positioning of the device within the flow path of a patient. These markers are visible under radiographic equipment. Other markers, such as gold, may also be provided, as will be apparent to a person skilled in the art. The markers are preferably attached to the portion of the device not in the flow path (e.g. region 220).

In a preferred embodiment, the flanges are manufactured from a radio-opaque or magneto-opaque material. The flanges have fewer constraints as far as size, thickness and material than the remaining (the porous section of the device that is); the flange thickness may be on the order of 100-200 microns so that they can be well-visualized under an x-ray beam or within a magnetic field. It is also beneficial to manufacture the device such that the flange is the portion which is visualized during implantation because the edges, or limits, of the device which engage ad attache to the second flow path will be the most important part of the device for the operator to visualize during the implantation.

Methods of Implanting the Device

FIGS. 3A-3C illustrate a method of folding and reducing the filtering device prior to deployment. Device 300 begins in an expanded or deployed state (FIG. 3A) with flanges 310 and 312 unfolded outward and the device substantially planar. FIG. 3B shows substantially planar device 300 in a partially reduced state such that a delivery sheath (not shown) may be used to deliver the device; in this case, flanges 310 and 312 are folded against the substantially planar portion of the device. The device can be further reduced through bending, as is shown in FIG. 3C, in order to better fit into a delivery catheter. Alternatively, reduced device 300 may be manufactured with a natural curve, as illustrated in FIG. 3C, to fit within a cylindrical delivery sheath, again with inwardly folded flanges 310 and 312. The general requirement is that the flanges be substantially parallel to the filter when in the reduced state inside a delivery catheter and substantially non-parallel when in the deployed state within a second flow path. When the delivery sheath is removed, as discussed in detail below, device 300 expands to a deployed delivery profile and flanges 310 and 312 fold outward to engage the walls of the second flow path. The size and shape of the device is preferably chosen by the operator to match the inlet of the flow path as will be further explained below.

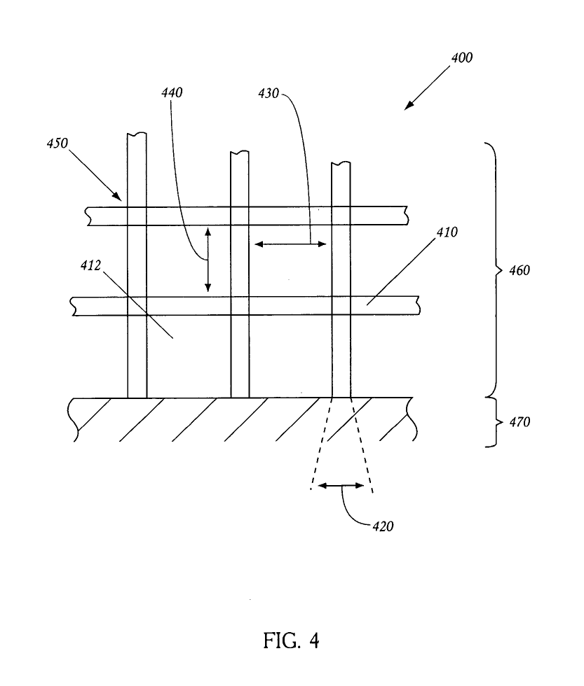

FIG. 4 depicts a detailed view of an embodiment of a region of the device of the current invention 400 showing struts 410 and openings (pores) 412 which can serve as openings for blood flow, but effectively prevent or divert undesirable embolic material flowing in the blood from entering the second flow path, as is described in detail below. In one embodiment, the device has openings (i.e. pores) 412 between the struts defined by dimensions 430 and 440, which are sized such that the smallest dimension of spacings 430, 440 is several fold larger that the maximal strut cross-section 420. In certain embodiments, the smallest dimension of spacings 430, 440 is at least the 5-20 fold, more preferably 20-50 fold, and even more preferably 50-100 fold larger than the maximal strut cross-section 420. The maximal strut cross-section refers to the largest possible dimension of an individual strut when viewed in cross-section. The relative size of the openings 412 compared to the strut cross-sections ensures that there will be minimal resistance to blood flow through the device which in turn ensures that there will not be overgrowth of endothelial cells from the edges of the flow paths.

In certain embodiments, openings (pores) 412 are greater than 50 times larger than the maximal cross-section 420 of the struts surrounding the pore. The larger the ratio between the maximum pore dimension and the maximum strut dimension, the lower the resistance will be to flow; e.g. from the first flow path to the second flow path.

The largest of pore dimensions 430, 440 dictates the largest particle which can be pass through the pore while the porosity index and the specific porosity index define the overall drag imposed on the fluid portion flowing through the device. In one embodiment of the invention, e.g. when device 400 is placed at the interface of a flow path which continues on to the cerebral circulation, openings (pores) 412 in device 400 should be no larger than 150 microns in any or all dimensions. This is preferable as particles larger than 150 microns have been shown to cause clinically relevant strokes. Preferably, openings 412 are about 100 microns in maximum dimension to allow an even larger margin of safety with respect to the particles which may flow through device 400. Device porosity may vary according to the actual conditions associated with embolic material of different patients and in different disease states. Smaller pore size leads to greater resistance to flow through the filter; however, this tradeoff can be partially offset by a smaller strut cross-section and therefore a pore:strut cross-sectional ratio equivalent to larger pore sizes.

Strut crossover region 450 in FIG. 4 is that region of the struts where there is overlap between struts traveling in opposite directions. Region 450 is very important in some embodiments because it is a direct consequence of the methods used to manufacture the device. Furthermore, depending on the nature of this overlap (e.g. non-continuous), a protected nidus for emboli generation may exist. In one embodiment, the overlap region 450, that is, the change from the vertical to the horizontal can be continuous . . . such would be the case if the device was directly cut from stock material (e.g. laser) or if a mold were used to produce the device. Alternatively, the cross-over region can be truly overlapped and discontinuous if the filter were constructed through a weaving process well-known in the art.

In some embodiments, pores 412 (FIG. 4) are smaller than about 20 microns in diameter such that blood components cannot flow through the pores and such that endothelial coverage of the pores will occur. This is the case when the first flow path is the atrium, for example, and the second flow path is a patent foramen ovale or other defect in the heart; or when the second flow path is an aneurysm sac such as a cerebral aneurysm.

As noted above, device 400 may be formed from one region without distinction between the flow path portion and the non-flow portion as far as structure and/or porosity. Alternatively, as shown if FIG. 4, device 400 comprises one or more regions 460, 470. As illustrated in FIG. 4, region 460 is the filter, and region 470 functions to maintain the device at a vessel opening by promoting healing around region 470 or by stabilizing the device within the first flow path, as described above.

Device Positioning

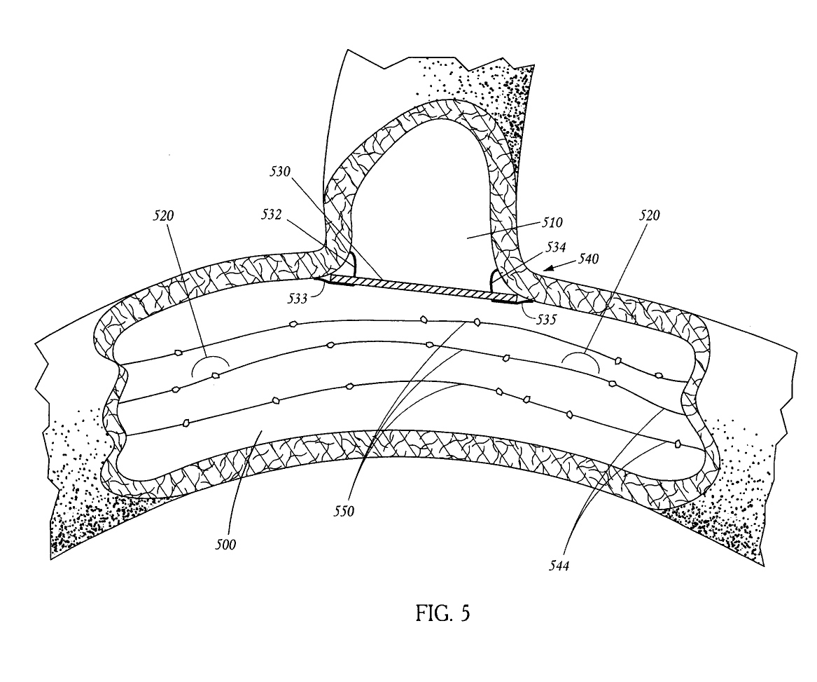

FIG. 5 is an illustration of the device in its operative position; depicted, is first flow path 500 and second flow path 510 of a patient. Device 530 is held in place by flanges 532, 534 and optionally, 533 and/or 535. Blood, generally referenced by 550, flowing throughout first flow path 500 is indicated in FIG. 5 by the space between all other designated flow paths, filtering device elements, and components. FIG. 5 shows the device of FIG. 2A in position at the branch point of first and second flow paths 500 and 510 with flanges 532 and 534 in an intermediate position between perpendicular and parallel to the device surface and securing the device in place by creating a frictional force on the wall of the second flow path and optionally, the first flow path.

Using suitable imaging equipment, filtering device 530 is inserted through the vasculature of a patient in a compressed, undeployed configuration (depicted below) into the first flow path 500, until device 530 is positioned at the branch zone 540, with the filtering element 530 extending across the inlet to the second flow path 510. The device is then allowed to expand to a deployed, uncompressed configuration in the operative position (shown and described further below). The catheter is then removed via the vasculature of the individual and deployment of the filtering element 530 completed, as illustrated in FIG. 5. In this position, embolic material, which is schematically illustrated as particles 520 flowing along flow lines 550 in FIG. 5, flow in first flow path 500 and upon meeting filtering device 530, are prevented from entering second flow path 510, because their size is larger than the device openings (pores) of filtering device 530; they are thus filtered, or deflected away from second flow path 510. Embolic material 544 may originate, e.g., in the heart or the aorta.

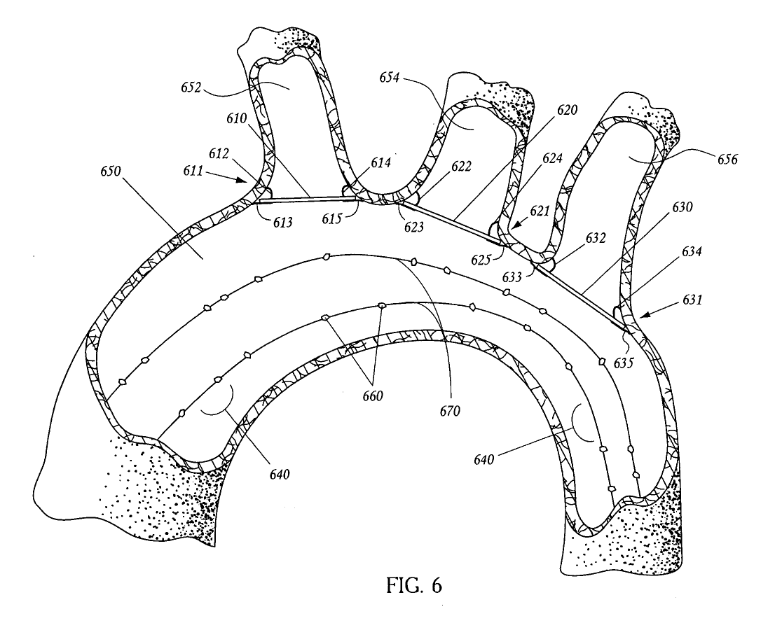

In certain embodiments of the invention, flow path 510 is a carotid artery, vertebral artery, brachiocephalic artery, or renal artery. The filtering of embolic material 520 from the blood flowing into the second flow path 510 prevents the emboli from possibly occluding smaller diameter flow paths located downstream from the treatment site as is found in all organs. If the procedure is being performed at the interface of the aorta and the carotid artery, the device can possibly prevent emboli from reaching the brain of a patient and can therefore FIG. 6 shows several devices in operative position . . . the devices are similar to FIG. 2A in position in the branch zone of first flow path 650, second flow path 652, third flow path 654, and fourth flow path 656. Devices 610, 620 and 630 are shown in an operative position and are independent of one another. In this example, an aortic arch is depicted and the second flow paths of interest 652, 654, and 656 are the right brachiocepahalic, the left common carotid, and the left subclavian arteries, respectively. Device 610 is held in place by flanges 612, 614, (and optionally) 613 and/or 615. Likewise, device 620 is held in place by flanges 622, 624, (and optionally) 623 and/or 625. Device 630 is held in place by flanges 632, 634, (and optionally) 633 and/or 635. Blood, generally referenced by 640, flowing throughout first flow path 650 is indicated in FIG. 6 by the space between all other designated flow paths and filtering device elements and components. The flanges and specific structural and material components of the overhang regions (not depicted) allow for chronic implantation of the devices.

Using suitable imaging (e.g. fluoroscopy, ultrasound, magnetic resonance, etc.) equipment, filtering devices 610, 620 and 630 are inserted through the vasculature of a patient into first flow path 650 until the filtering devices are positioned within branch zones 611, 621 and 631. When the devices are in these positions, embolic material, which is schematically illustrated as particles 660 flowing along flow lines 670 in FIG. 6, flow within first flow path 650. Upon meeting any of devices 610, 620 and/or 630, the particles are prevented from entering second, third and fourth flow paths 652, 654 and 656 because the size of the embolic material is larger than the device openings, the pores. The particles are thus filtered away from second, third and fourth flow paths 652, 654 and 656. It is also possible that the particles are broken up by the struts of the device to a size which does not cause damage to the end organ, in this case the brain. The devices of this embodiment of the invention are particularly well-suited for the prevention of stroke.

Method of Implantation

The operation of the filtering device of the invention will now be discussed in reference to device 300, having flanges 310 and 312, and optionally, flanges 314 and 316, of FIGS. 3A-3C for the sake of clarity, and not by way of limitation. It will be appreciated that other embodiments of the invention can be used and delivered in a similar manner.

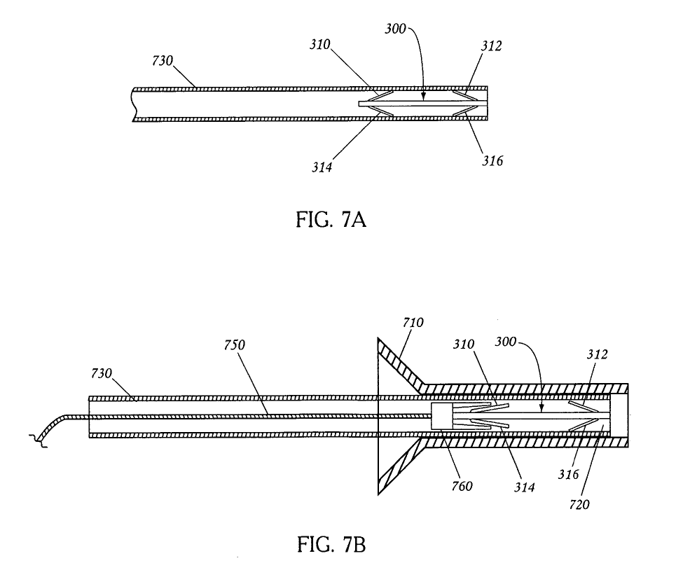

The filtering device can be obtained from the manufacturer already pre-loaded at the distal end of catheter 730 as illustrated in FIGS. 7A-7B; alternatively, the device is loaded into the catheter by the physician just prior to the procedure. The catheter and filter form a device in accordance with another aspect of the invention. The device includes a pusher wire 750 for advancing the filter into and out of the lumen (during deployment for example) of the catheter, as described below.

Device 300 contacts end piece 760 mounted on the distal end of pusher wire 750. The contact between the device 300 and endpiece 760 is a reversible one and can be designed to be reversible via electromagnets, heat deformable nickel-titanium, thermo-deformable polymers, an electrochemically degradeable part, a mechanical interlock, or a friction contact, etc. Pusher wire 750 is provided with the filter and catheter assembly by the manufacturer and is provided to the operator pre-loaded at the distal end of the catheter.

FIG. 7B depicts catheter 730 being placed into a sheath 710. As is known to those skilled in the art, sheath 710 is a holding channel (within a large vessel such as the femoral artery) for placement of devices such as catheters into and out of the vasculature of a patient. As is well-known in the art, the sheath maintains the access path for the devices while different devices are fed into and out of blood vessels such as the femoral or brachial arteries. As discussed above, the filter is positioned within a patient to prevent emboli from reaching a second flow path from a first flow path.

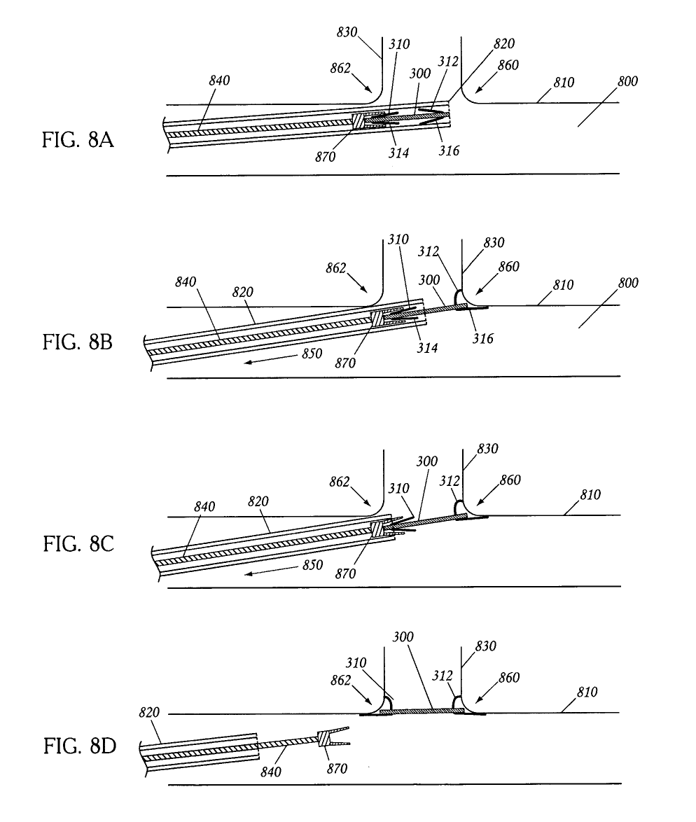

The catheter is now advanced to the region of interest within the patient as illustrated in FIGS. 8A-8D using filter 300 of the invention. In accordance with the method, a loaded catheter 820 is guided within the lumen 800 of first flow path 810 to the inlet of second flow path 830 (FIG. 8A) using standard imaging techniques, e.g., fluoroscopy, MRI, CT scan, etc. Optionally, the catheter, loaded with the device, is delivered to the region of interest over a guiding wire (not shown) and through a second channel in the catheter or pusher wire (not shown).

Preferably, pusher wire 840 is held steady and the catheter 820 is retracted in a direction indicated by arrow 850 to partially release filter 300, which allows flange 312 to unfold and frictionally engage the wall of the bifurcation zone, thereby securing the distal end of filter 300 against the wall of the inlet of first and second flow paths 810 and 830. As the device 300 is released from the catheter, the filter may be held in place near or against the wall of the branch zone by end piece 870 of pusher wire 840. While partially outside of the catheter (FIG. 8C), the filter may be positioned as desired by manipulating pusher wire 840.

During the final stage of deployment, pusher wire 840 is held steady and the catheter is retracted, as shown by arrow 850, to fully expel the filter (FIG. 8D) from the catheter, allowing flange 310 to engage the lumen of second flow path 830. The filter is released from the pusher wire by one of the reversible mechanisms described above. The filter expands to a predetermined or desired dimension such that a substantial (i.e. high percentage) of the inlet from first flow path 810 to second flow path 830, is effectively covered. Preferably, the filter has a substantially planar shape and lies flush against the inlet to second flow path 862. In certain embodiments of the invention, the dimensions of the filter are selected such that in an expanded state, a portion of the device extends beyond the circumference of the inlet to the second flow path. Such extension can facilitate chronic implantation or may be used to cover more than one flow path.

A preferred filter of the invention effectively covers at least 90% of the inlet to the second flow path with the filter portion (130 in FIG. 1), preferably at least 95%, more preferably at least 99%, and even more preferably 100% of the inlet to the second flow path. Despite “covering” 100% of the vessel ostium, because of its porosity index, the filter effectively blocks only about 25% of the area of the ostium because of the porous nature of the filter. More preferably, the filter blocks less than 10% of the area of the ostium and even more preferably, the filter blocks less than about 5%. Thus, the device, when in an operative position, is effective to filter (deflect) at least a portion of emboli which are clinically relevant (>100 microns in a preferred embodiment) in the first flow path away from the second flow path. Preferably greater than about 40%, more preferably greater than about 50%, and even more preferably between 75% and 100% of embolic material greater than a clinically relevant size (typically 100 microns) is prevented from entering the second flow path.

The relatively small, planar dimensions of the filter with overhang regions and flanges adapted to secure the device (chronically) at the interface of the first and second flow paths respectively are useful in treating targets located in tortuous and narrow vessels, for example in the neurovascular system, certain sites within the coronary vascular system, or in sites within the peripheral vascular system such as superficial femoral, popliteal, or renal arteries. In certain embodiments of the invention, the first flow path is the aortic arch of a patient, and the second flow path is the right brachiocephalic artery, the left common carotid artery, or the left vertebral artery.

The invention is particularly well-suited for reducing the risk of stroke in a patient by positioning the filter which has been configured and dimensioned for implantation in the patient's common carotid artery as it branches from the aorta. In this embodiment, the filter is configured and dimensioned such that once it has been positioned in the patient, it substantially covers the inlet of the common carotid artery as the artery branches from the aorta. The filter has openings, described above, which are sized and configured to prevent emboli in the blood from entering the common carotid artery without blocking (i.e. substantially reducing) blood flow through the common carotid artery. In some embodiments, the pressure drop across the filter is less than 5 mm HG when the pressure in the first flow path is between 80 mm and 150 mm HG. More preferably, the pressure drop across the filter is less than 1 mm HG when the pressure in the first flow path is between 80 mm and 150 mm HG. In a preferred embodiment, the vertebral arteries and the common carotid arteries all receive a device in order to prevent embolization to all vascular territories of the brain.

Coating

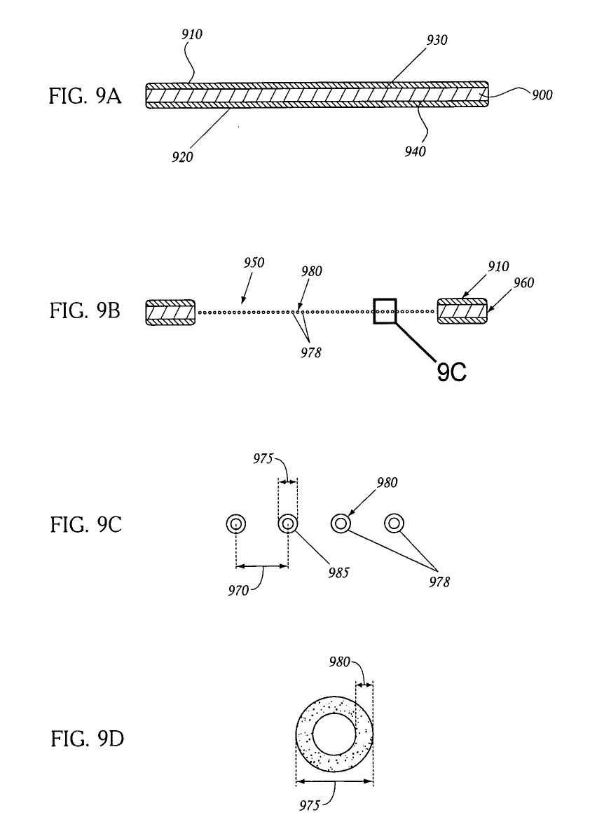

FIG. 9A depicts a longitudinal view of device 900 viewed from the side. According to one embodiment of the invention, device 900, which is similar to the device illustrated in FIGS. 1A-1C, has biocompatible coatings 910 and 920 disposed on first and second surfaces 930 and 940 of device 900. The biocompatible coatings may be covalently or non-covalently bound such that the biocompatible coatings impart one or more desirable properties to device 900. For example, certain biocompatible coatings may be added to the device in order to reduce locally turbulent blood flow near, against, or through the device. Preferably the coating is not greater than 0.5 microns in thickness and more preferably the coating is not greater than 100 nm thick. Even more preferably, the coating thickness is not greater than the thickness of one molecule.

The biocompatible coating can be applied to the entire device or to a portion of the device; alternatively, different coatings can be applied to different regions of the device. For example, one region can have a coating applied which is adapted for tissue ingrowth (e.g. the overhang portion 220 in FIG. 2.) Another region, such as the porous region 240 in FIG. 2, can have a distinct coating applied to the struts which induces its own desirable effect (e.g., promotion of laminar flow) on the blood flowing through the pores.

FIG. 9B depicts a longitudinal view through the porous section of the device (130 in FIGS. 1A-1B). 950 is the section through which blood flows and which filters or deflects emboli from the blood. 960 is the section adapted to secure the device to the wall of the first flow path (140 in FIGS. 1A-1B) and 910 is an example of a coating discussed with reference to FIG. 9A, which is adapted to induce healing and to secure the overhang portions of the device to the first flow path. 970 is the distance between struts 980 which defines the pore, or the hole size between struts 980.

FIG. 9C is a magnified view of the porous region of the device (240 in FIG. 2). 985 depicts the coating on the struts which, in contrast to the coating applied to the overhang region, can be adapted to minimize the friction between the strut and fluid flow; such is the case, for example, with a hydrophobic coating.

970 is the distance from the center of one strut to the center of an adjacent strut. This “interstrut” distance anywhere along the portion of the device within the blood flow path 950 is preferably at least five times greater than the maximal strut dimension 975, and more preferably at least 10 times greater and even more preferably, 50 times greater than the maximal strut dimension 975 to avoid an undesirable amount of resistance to blood flow across the device.

FIG. 9D is a further magnified view of the struts, which as discussed above, are preferably smaller than about 20 microns in diameter, and more preferably, smaller than about 5-10 microns, and even more preferably, smaller than 0.5 to 2 microns in diameter. Dimension 975 is the maximum strut diameter, inclusive of a coating specific to the struts. Although a circular strut formation is shown, the strut can have a characteristic cross-section, typically the maximal dimensional cross-section of the strut. Coating 980 is preferably not greater than 10% of the strut thickness, or about 1-2 microns; more preferably, the coating is not greater than about 1% of the thickness of the strut, or about 100-200 nanometers in thickness. More preferably, the coating is not greater than 50-100 nm in absolute thickness. Even more preferably, the coating is the thickness of one molecule.

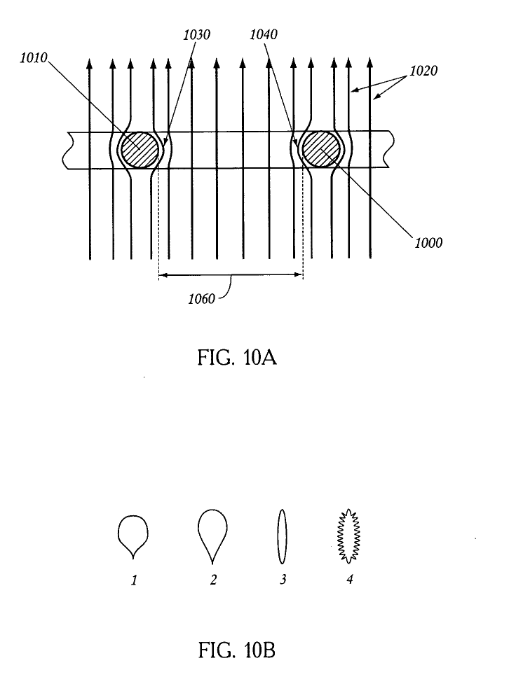

FIG. 10A illustrates a cross-section of two adjacent struts 1000 and 1010. As blood 1020 flows over the struts, the boundary layer 1030 created around one strut 1010 does not interfere with the boundary layer 1040 of an adjacent strut 1000. As noted above, preferably there is at least a five-fold separation 1060 between the boundary layers of adjacent struts; such a spatial relationship prevents the creation of turbulent flow (i.e. maintains laminar flow); such a spatial relationship further maintains a low resistance to flow between the struts and across the surface of the device.

FIG. 10B illustrates various strut designs adapted to decrease the thickness of boundary layers 1030 and 1040 and induce laminar flow over the struts, or otherwise decrease the drag force on the struts. FIGS. 10B1-2 are tear drop shaped strut designs which are adapted to allow laminar blood flow over each strut such that the boundary layer is minimized by creating an aerodynamically favorable environment around the strut (i.e. similar to an airplane wing). FIG. 10B3 (and 10B2 as well) is an example of an ovoid shaped strut which creates a shearing effect on the emboli as the emboli pass through the device or are deflected by the device. FIG. 10B4 is an example of a dimpled strut discussed above. Such dimpling has the effect of reducing the overall drag, or resistance to flow, on the device; such drag reduction is well known in the art of producing golf balls.

As illustrated in FIGS. 10A-B, the struts do not have biocompatible coatings. However, as noted above, in additional to proper strut spacing, a biocompatible coating may be applied to the struts in order to further reduce turbulence and enhance laminar flow. Preferred coatings include but are not limited to polytetraethylfluorine (PTFE), polyvinylfluoridene (PVDF), and polyalilene, etc.; because these coatings are highly hydrophobic, they will likely decrease the degree of friction between the blood and the surface of the device. Newer surface coatings from the evolving field of MEMS (microelectromechanical systems) and NEMS (nanoelectromechanical systems) will provide even further surface enhancements to reduce friction between the struts and the blood flow.

Biocompatible coatings may also be useful to reduce adverse tissue reactions or induce preferred tissue reactions. These coatings may be placed on all or part of the device; furthermore, different biological reactions will be desired on different portions of the device. For example, tissue growth will be preferred over the flanges and over the portion of the device which contacts the walls of the first flow path but is not doing the filtering or deflecting; in this case, a coating which encourages fibroblast ingrowth (e.g. TGF-beta, or a tissue irritant) or endothelial ingrowth (e.g. VEGF) may be preferred.

The coatings or films may also be used to administer a pharmaceutically active material to the site of the device placement. Generally, the amount of coating to be applied to the device will vary, depending on, among other possible parameters, the particular materials used to prepare the coating, the design of the device, and the desired effect of the coating.

It is important to note that the medical device of the invention may be coated with coatings that comprise drugs, agents or compounds; or the coating may be a simple one which does not contain drugs, agents or compounds. The entire medical device may be coated, a portion of the device may be coated, or no portion of the device may be coated. The coating may be uniform or non-uniform. The coating may also be discontinuous. However, in embodiments of the invention described above where markers are present on the device, the markers are preferably coated in a manner so as to prevent coating buildup which may interfere with the operation of the device.

In certain embodiments, the thickness of the coating may comprises from about 0.1 to about fifteen percent (by area) of a given cross-section of the device; and preferably, from about 0.4 to about ten percent weight. The coatings may be applied in one or more coating steps depending on the amount of material to be applied. For example, different polyfluoro copolymers may be used for different layers in the device coating. In certain exemplary embodiments, it is highly advantageous to use a diluted first coating solution comprising a polyfluoro copolymer as a primer to promote adhesion of a subsequent polyfluoro copolymer coating layer that may include pharmaceutically active materials. The individual coatings may be prepared from different polyfluoro copolymers.

Additionally, a top coating may be applied to delay release of the pharmaceutical agent or it could be used as the matrix for the delivery of a different pharmaceutically active material. Layering of coatings may be used to stage release of the drug or to control release of different agents placed in different layers.

Blends of materials, such as polyfluoro copolymers, may also be used to control the release rate of different agents or to provide a desirable balance of coating properties, i.e. elasticity, toughness, etc., and drug delivery characteristics; for example, release profile. Polyfluoro copolymers with different solubilities in solvents may be used to build up different polymer layers that may be used to deliver different drugs or to control the release profiles of a given drug. As will be readily appreciated by those skilled in the art, numerous layering approaches may be used to provide the desired degree of drug delivery.

The amount of therapeutic agent is dependent upon the particular drug employed and the medical condition being treated. Typically, the amount of drug represents about 0.001 percent to about seventy percent of the total coating weight; more typically, the amount of drug represents about 0.001 percent to about sixty percent of the total coating weight. It is possible that the drug may represent as little as 0.0001 percent of the total coating weight.

The quantity and type of materials employed in the coating film comprising the pharmaceutic agent will vary depending on the desired profile and the amount of drug employed. The product may contain, for example, blends of the same or different polyfluoro copolymers having different molecular weights to provide the desired release profile or consistency to a given formulation. See, e.g., U.S. Pat. No. 6,790,228, issued Sep. 14, 2004, which is incorporated herein by reference.

Polyfluoro copolymers may release dispersed drug by diffusion. This can result in prolonged delivery of effective amounts of the drug. The dosage may be tailored to the subject being treated, the severity of the affliction, the judgment of the prescribing physician, and the like.

Individual formulations of drugs and copolymers may be tested in appropriate in vitro and in vivo models to achieve the desired drug release profiles. Methods for the coating of substrates for pharmaceutical use are well known in the art, and described, e.g., in U.S. Pat. No. 6,783,768, issued Aug. 31, 2004, which is incorporated herein by reference.

Additional desirable biocompatible coatings and methods of coating the devices of the present invention are described in U.S. patent application 2003/0113478, published Jun. 19, 2003, which is incorporated herein by reference.

Alternative Designs

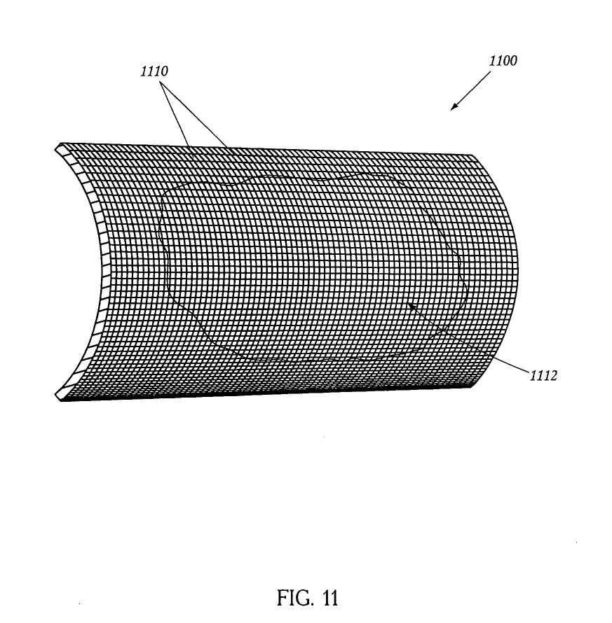

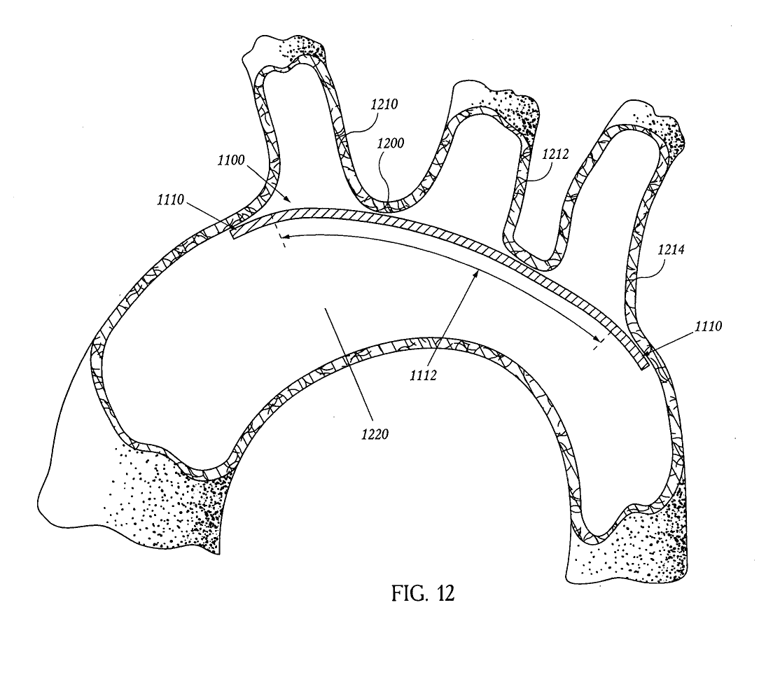

In certain embodiments, a single device is configured to be implanted in the patient to substantially cover more than one opening. For example, as illustrated in FIGS. 11 and 12, a substantially two dimensional device 1100, which contains struts 1110, 1112 (FIG. 11), and which has been configured to fit over more than one flow path is implanted in a permanent fashion along the wall (FIG. 12) 1200 of aortic arch 1220. The device is flush with the inlets of arch vessels 1210, 1212 and 1214; such an implantation configuration will prevent embolic material greater than 100 microns, and preferably greater than 50 microns, from reaching the brain of the patient while leaving blood flow to the brain substantially unaffected. Region 1110 is adapted for chronic implantation in a blood vessel by one of the techniques above (a coating or specific pore size to induce tissue ingrowth); blood does not flow through this region. Points 1200 and 1205 depict regions of the device where the device is additionally secured to the aortic arch. In one embodiment, the device is “spot welded” to the aorta using an activateable curing agent such as an agent which is activateable by ultraviolet light or other light source which can induce cross-linking and bonding of material surfaces. The activateable agent can be applied after the device is in place along the aorta or before the device is placed along the aorta. Of course, the activating light source is applied after the device is placed along the aorta.

In other embodiments, the current inventive device forms part of a second device. For example, stents are well known in the art of vascular devices. Stents are held in blood vessels by a frictional force imparted on the blood vessel by the stent. The current inventive device can be added as a component of a stent in the case where a stent is used to partially block and/or some or all of its components from reaching a second flow path.

Device Alterations to Allow Access to the Distal Vasculature

In certain situations, it may be desirable for the physician to reach the distal vasculature protected by the device. For example, it may be desirable, in the case when a stroke does occur even when the device is placed over the takeoff of the carotid artery (for example), to access the distal cerebral vasculature with a diagnostic or therapeutic catheter and device. In certain embodiments, a region of the flow path portion of the device has a second defined region, which is, e.g., 1-2 mm in diameter, and allows for reversible passage of a guide catheter or wire.

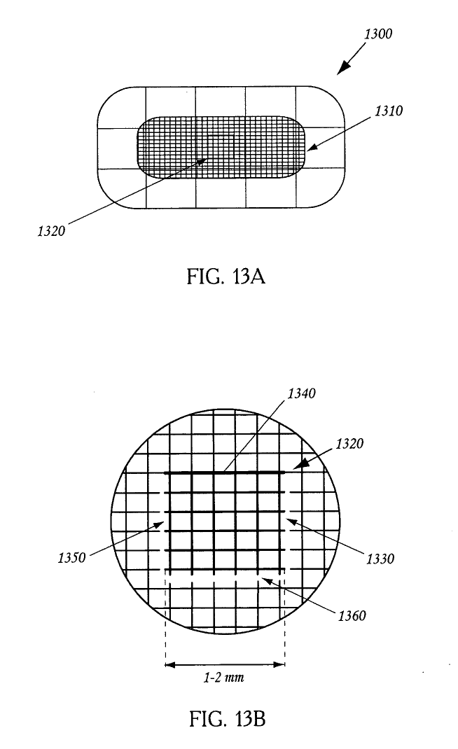

FIG. 13 depicts one such embodiment. 1310 depicts the flow path region corresponding to region 130 in FIG. 1A. 1320 depicts the region which is accessible to a catheter. FIG. 13B depicts a magnified view of region 1320 in which 1330 and 1360 depict dissociated struts which will allow a pivot at region 1340 when a catheter is urged through window 1320. Alternative embodiments involve creation of true joints at region 1320 to allow window 1320 to open with ease and return to a pre-opening location with ease. Alternative embodiments embody strut continuity at regions 1330,1360, and 1350 rather than discontinuity. Such alternative embodiments involve weak links between at locations 1330,1360, and 1350. Such links can be created after manufacture of the device by cutting what had previously been a continuous strut, then bonding the struts by a weaker material such as a polymer material.

Methods of Manufacture

The device may be manufactured by a number of methods known in the art. Exemplary methods of manufacturing the device by laser micromachining and chemical and electrochemical etching are described in Examples I and II below.

Other manufacturing methods include the use of photolithography to pattern the device. In this method, a light blocking or light transparent mask is used to pattern a material, such as silicon or a polymer. Light is used to specifically activate the region not covered by the mask. Subsequently, an etching process is used to etch the material which was not exposed to the light source which will lead to a mesh with a specific porosity, porosity index, and specific porosity index. Typically strut sizes from 1-5 microns can be produced using such a method and porosity indexes and specific porosity indexes ranging from 80-99% or from 85-95%.

Other methods such as nano-imprint lithography, micromolding in capillaries, nanomolding, and soft lithography can also be used to produce the device of the present invention. For example, U.S. Pat. No. 5,820,769 (herein incorporated by reference in its entirety) teaches the use of electron beam (e-beam) lithography to create nanometer sized magnetic elements in a substrate. PMMA is applied to a silicon or gold substrate and then a micro or nano pattern of dots is created in the PMMA using an electron beam followed by a solvent developer which etches the regions exposed to the electron beam. The etched regions are then filled in with a metal in a dot pattern. In the context of manufacturing the device of the current invention, a mesh with a desired nano or micro porosity, porosity index, and specific porosity index can be manufactured by etching the desired pattern with an electron beam; subsequently, the device is created by filling the etched pattern with electrochemically deposited material. With such an electron beam, or nano-imprint process, the filter of the current invention can have a porosity index of 95-99.5% with the porosity of individual pores remaining in the range of 50-150 microns. Such a configuration would be highly beneficial in preventing emboli from traveling into the second flow path while freely allowing flow of the blood components through the device.

U.S. patent application 2003/0170996 (herein incorporated by reference in its entirety) describes an embossing technique to produce dot like structures. In this process, a pattern is imprinted in a polymer substrate, after which the pattern is developed to completion using reactive ion etching. A filter device of the present invention can then be deposited into the pattern on the substrate.

Vapor deposition processes have also been used to produce medical materials. U.S. patent application 2002/0165600 (herein incorporated by reference and in its entirety) teaches the use of physical and chemical vapor deposition as well as sputtering techniques to put down thin film layers on devices such as guidewires. Etching processes as described herein can then be used to create meshes with defined porosities, porosity indices, and specific porosity indices as described herein. Chemical and physical vapor deposition as well as sputtering processes can be also be used to improve the physical properties of an already created filter (e.g. a filter created from a laser process or a nano-imprint process).

A further example of a vapor deposition process to create a medical device is described in PCT/US01/02253 (herein incorporated by reference in its entirety) in the context of creating nickel-titanium devices (e.g. a stent). A magnetron sputtering technique is used to coat a mandrel with a material after which the material can be patterned through a photolithography technique with subsequent etching of the unexposed regions of the device.

### EXAMPLES

I. Laser Micromachining

A substantially planar embolic filter was produced generally following the following laser cutting process. Using stock 25 micron stainless steel sheet metal, a 355 nm q-switched Nd:YVO4 laser system was used in conjunction with a direct write software program to produce a filter with 125-150 micron pores and 15-25 micron strut diameters. The struts were substantially ovoid and the diameter was consistently found to be in the range of 15-25 microns. The 25 micron thickness was amenable to folding of the device into a sheath. The porosity index of the device is between 75 and 78%.

A similar methodology was used to produce a filter with 10-15 micron strut diameters and pore sizes from 125-150 microns. The porosity index of this filter is approximately 79-83%.

II. Chemical and Electrochemical Processing

To further decrease the size of the struts and therefore increase the porosity of the filter and without having to deviate from the laser based manufacturing process, the filter was placed into an acid bath consisting of a mixture of hydrofluoric acid and nitric acid in a 1:1 ratio as is well know to those skilled in the art. This processing step increased the porosity index to 85-95% depending on the etching time.

In another manufacturing process, the device was exposed to an electric current while in the acid bath, which leads to controlled etching of the struts to further increase the porosity of the device without deviating from the laser manufacturing method above.

In another manufacturing process, e-beam lithography is used to create a pattern and an electrochemical process (e.g an electroplating or an electroless processes) is used to create structures within the patterns (as described above) after which the pattern is removed.

The above described methods are useful for the filtering portion of the current invention. Although the methods can be used for other portions of the invention (e.g. the flange or the overhang region), they do not necessarily have to be used for other portions of the invention. In one embodiment, the filtering portion is produced using one or more of the methods above and flanges are subsequently welded on. Alternatively, the filtering portion serves as a component of a larger device such as a stent, filter, or a device to unload an aneurysm.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A device adapted for interposition between a first flow path and at least one second flow path, the device comprising;
(i) a first surface facing toward an opening of the at least one second flow path; and
(ii) a second surface facing away from the opening of the at least one second flow path;
wherein the device, when in operative position, extends less than the complete circumference of the first flow path and substantially covers the opening of the at least one second flow path; and
wherein at least one portion of the first surface or second surface is adapted for chronic implantation of the device.

2. The device of claim 1, wherein the device is substantially planar.

3. The device of claim 1, wherein the thickness between the first surface and the second surface is less than about 25 microns.

4. The device of claim 1, wherein at least a portion of the device is porous and said porosity is characterized by a porosity index between about 80% and 99%.

5. The device of claim 1, further comprising one or more coatings disposed on at least one or more portions of the first and/or second surfaces.

6. A medical device comprising;
a filtering region and a non-filtering region;
wherein the filtering region is characterized by struts which define pores and which are characterized by a largest cross-sectional area;
and wherein the filtering region is further characterized by a porosity index and a specific porosity index of at least 70% and the largest cross-section of the struts is smaller than about 50 microns.

7. The medical device of claim 6 wherein said largest cross-section of said struts is smaller than about 25 microns.

8. The medical device of claim 6 wherein said largest cross-section of said struts is smaller that about 10 micron.

9. The medical device of claim 6 wherein said largest cross-section of said struts is smaller than 1 micron.

10. The medical device of claim 6 wherein the size of said pores is greater than 2500 square microns.

11. The medical device of claim 8 wherein the minimum distance between substantially parallel said struts is at least 100 microns.

12. The medical device of claim 6 wherein said porosity index and said specific porosity index are greater than about 80%.

13. The device of claim 6 wherein said porosity index and said specific porosity index are greater than about 90%.

14. The device of claim 6 wherein said porosity index and said specific porosity index are greater than about 99%.

15. The medical device of claim 6 wherein the filtering or non-filtering portion is further adapted to act as a baffle for fluid flow over its surface.

16. The device of claim 6 wherein said non-filtering portion is shaped like a stent.

17. The device of claim 6 wherein said non-filtering portion is adapted for placement in the vena cava.

18. The device of claim 6 wherein said filtering portion is further configured to act as a baffle in fluid flow.

19. The device of claim 6 wherein said filtering portion is substantially planar and said non-filtering portion is substantially planar.

20. The device of claim 6 wherein at least a portion of said filtering portion is comprised at least in part from a vapor deposited material.

21. The device of claim 6 wherein at least a portion of said filtering portion is comprised at least in part from a material formed from a process involving an electron beam lithographic step.

22. The device of claim 6 wherein at least a portion of said filtering portion is comprised at least in part from a material formed from an embossing process.

23. The device of claim 6 wherein at least a portion of said filtering portion is derived from an electrochemical process.

24. The device of claim 6 wherein said filtering portion and said non-filtering portion are attached by a weld.

25. The device of claim 6 wherein at least one of the non-filtering and filtering portions further comprises a covalently bond organic molecule.
